(19) United States
(12) Patent Application Publication
(10) Pub. No.: US 2010/0131009 A1
(43) Pub. Date: May 27, 2010

(54) SPINOUS PROCESS IMPLANT SPACER AND METHOD OF USE THEREFOR

(76) Inventors: CHRISTIAN ROEBLING, Freiburg (DE); Rolf Ackermann, Rietheim-Weilheim (DE)

Correspondence Address: LACKENBACH SIEGEL, LLP, LACKENBACH SIEGEL BUILDING, 1 CHASE ROAD, SCARSDALE, NY, 10583, US

(21) Appl. No.: 12/622,076

(22) Filed: Nov. 19, 2009

(30) Foreign Application Priority Data
Nov. 19, 2008 (EP) 08 020 138.7

(51) Int. Cl. A61B 17/70; A61B 17/88

(52) U.S. Cl. 606/249; 606/279

(57) ABSTRACT

A spinous process implant, and a method therefor can be positioned with its longitudinal axis substantially perpendicular to the median sagittal plane of the patient, between an upper and an adjacent lower spinous process. The implant comprises, as spacers, upper support means, and lower support means, which, in each case, are applied with a recess saddle against the upper or lower spinous process. The support means can be spread apart in each case by means of clamping means, which are arranged between the support means in the longitudinal direction.

### CROSS REFERENCE TO RELATED APPLICATIONS

This application is related to, and claims priority from, European Patent Application Serial No. 08 020 138.7, filed Nov. 19, 2008, the entire contents of which is incorporated herein fully by reference.

### BACKGROUND OF THE INVENTION

1. Field of the Invention

The present invention relates to a spacer for facilitating a spinous process implant. More specifically, the present invention relates to a spinous process implant and spacer that can be implanted easily and is usable in many anatomical situations

2. Description of the Related Art

Degeneration of the vertebral column, which occurs particularly with increasing age, frequently leads to lumbar spinal canal stenosis. This is a narrowing of the spinal canal and of the intervertebral foramina (foramina intervertebralia), generally as a consequence of spinal disk degeneration. Typical symptoms are nerve pains in the back and in the legs, and in severe cases paralysis symptoms in the legs.

In simple cases, treatment with drugs and physiotherapy can be successful. In severe cases, distraction of the spine causes a decompression of the nerve structure. For long-term therapy, it is possible in this case to use implants between the spinous processes of the affected vertebrae. These spinous process implants have in common the use of a spacer which is inserted between the spinous process of the upper cranial vertebra, and the spinous process of the adjacent lower caudal vertebra. The spacer is introduced here laterally between the spinous processes, where its longitudinal axis is arranged so it is substantially perpendicular to the median sagittal plane, i.e., to the middle plane of the body.

From U.S. Pat. No. 5,860,977, a spinous process implant is known in which the spacer is substantially a cylindrical body, which is inserted between the spinous processes, and protected against lateral shifting in its longitudinal axis by wings arranged on its two lateral ends. A unilateral insertion of the implant is not possible here, because the spacer with a wing is inserted from one side, and then the second wing has to be placed on the other side of the spinous process.

The company Kyphon sells a unilaterally insertable implant under the name of “Aperius.” This implant presents a tubular spacer with two thinned zones at a separation. The spacer is introduced unilaterally between the spinous processes, and then braced in the longitudinal direction, so that the thinning zones are deformed to radially protruding wings, which keep the spacer positioned on both sides of the spinous processes.

What is not appreciated by the prior art is that these spinous process implants share the feature that the spacer presents a predetermined diameter. Therefore, depending on the anatomical circumstances, spacers with different diameters have to be used. Consequently, on the one hand, the interspinal space has to be measured in a first surgical step, to be able to choose the fitting spacer. In addition, spacers in different sizes have to be available.

In EP 1 330 987 B1, a spinous process implant is described in which, as spacer, a closed spring band is used, which has essentially the shape of a figure eight that is symmetric with respect to the sagittal plane. The implant is secured with securing clips to the respective spinous processes. The implant thus fixes the spinous processes of consecutive vertebrae with respect to each other, so that bending of the vertebral column is now possible only within the narrow limits of the elastic deformability of the implant. Here too, the implant must be chosen in each case in a size that fits the anatomical requirements.

Accordingly, there is a need for an improved spinous process implant that can be implanted easily and is usable in many anatomical situations.

### ASPECTS AND SUMMARY OF THE INVENTION

An aspect of the present invention is to provide a spinous process implant that can be implanted easily and is usable in many anatomical situations.

The present invention relates to a spinous process implant that can be positioned with its longitudinal axis substantially perpendicular to the median sagittal plane of the patient, between an upper and an adjacent lower spinous process. The implant presents, as spacers, upper support means, and lower support means, which, in each case, are applied with a recess saddle against the upper or lower spinous process. The support means can be spread apart in each case by means of clamping means, which are arranged between the support means in the longitudinal direction.

The essential idea of the invention is to use, as spacer that is inserted between the spinous processes, not a body with a predetermined, fixed diameter, but a spacer which presents two support means that can be spread apart from each other. In an introduction position, the support means are positioned one against the other with a small separation, so that the spacer presents only a small diameter. After the introduction between the spinous processes, the support means are spread apart, so that their separation becomes larger. As a result, the upper support means are applied against the upper cranial spinous process, and the lower support means against the adjacent lower caudal spinous process. In this way, the spacer is able to adapt to the given anatomical circumstances, and there is no need for different spacers for different dimensions of the interspinal space.

Clamping means are used here to spread the support means apart. They are arranged between the support means in the longitudinal axis of the implant, i.e., in the axis that is perpendicular to the median sagittal plane, and they engage on both lateral ends of the support means. As a result, a unilateral introduction of the implant is possible. The implant can be introduced unilaterally through a percutaneous incision between the spinous processes, and then it is spread apart by the unilateral actuation of the clamping means, and positioned and fixed between the spinous processes. The support means present, in each case, a recessed saddle, which forms a receptacle for the given spinous process. When the support means are applied, in the spread state, against the respective spinous process, then they are positioned with this saddle over a peripheral area against the periphery of the spinous process, which results in securing the support means, and thus the entire implant, with positive locking against lateral shifting, i.e., against shifting in the longitudinal axis of the implant, perpendicularly to the sagittal plane. The implant therefore does not require additional wings or other measures to protect against lateral shifting with respect to the spinous processes. This simplifies the construction and facilitates particularly the surgical technique during the introduction of the implant.

In another embodiment of the present invention, the support means are designed as spring leaves whose lateral ends are pulled together by the clamping means, so that the spring leaves form an upward or downward convex bulge, and spread apart.

In another embodiment, the support means are designed as support plates, which, during the actuation of the clamping means, are swiveled out of their introductory position, upward or downward, so that their separation becomes larger. In a simple embodiment, it is possible here to connect by articulation in each case an upper and a lower support plate to the two lateral ends, whose mutually facing free ends are swiveled away from the longitudinal axis upward or downward.

In an embodiment that allows for easy handling, the clamping means present a telescopic design. By telescopically pushing the lateral ends together, they can be moved toward each other, to spread apart the support means. The telescopic pushing together can here occur unilaterally by means of a self-inhibiting thread. The clamping means can also be designed so they can be moved freely into each other telescopically, where the two telescope parts become latched in the respective spread position, against the counterpressure of the spinous processes. In a variant of the invention, the telescope parts can be shifted further into each other by means of a resetting force, to spread the support means further apart, for the purpose of adapting the implant to the increasing separation of the spinous processes, for example, in the case of bone resorption.

The above, and other aspects, features and advantages of the present invention will become apparent from the following description read in conduction with the accompanying drawings, in which like reference numerals designate the same elements.

### BRIEF DESCRIPTION OF THE DRAWINGS

FIG. 1 shows a side view of a first embodiment of the spinous process implant in the introduction position;

FIG. 2 shows a perspective view of the implant in the spread position;

FIG. 3 shows an additional perspective view of this implant;

FIG. 4 shows a longitudinal section of the first embodiment of the implant;

FIG. 5 shows a second embodiment of the spinous process implant in the introduction position;

FIG. 6 shows the second embodiment in the spread position;

FIG. 7 shows a perspective view of the second embodiment, where the support plates have been omitted;

FIG. 8 shows a partial view of FIG. 7;

FIG. 9 shows an additional partial view, in which a support plate is shown for illustration; and

FIG. 10 shows a detail view of an embodiment of the clamping means.

### DETAILED DESCRIPTION OF THE PREFERRED EMBODIMENTS

Reference will now be made in detail to several embodiments of the invention that are illustrated in the accompanying drawings. Wherever possible, same or similar reference numerals are used in the drawings and the description to refer to the same or like parts or steps. The drawings are in simplified form and are not to precise scale. For purposes of convenience and clarity only, directional terms, such as top, bottom, up, down, over, above, and below may be used with respect to the drawings. These and similar directional terms should not be construed to limit the scope of the invention in any manner. The words “connect,” “couple,” and similar terms with their inflectional morphemes do not necessarily denote direct and immediate connections, but also include connections through mediate elements or devices.

The purpose of the spinous process implant according to the invention is to keep the spinous process (processus spinosus) of two consecutive vertebrae, in most cases two lumbar vertebrae, separated vertically. For this purpose, the implant is introduced laterally between the spinous processes in the median direction, and positioned in such a way that the longitudinal axis of the implant is arranged substantially perpendicularly to the median sagittal plane of the patient, i.e., to the middle plane of the body. To simplify the description, the following indications refer to the implanted position of the implant. Thus, the terms “upper” or “lower” refer to the cranially or caudally directed sides of the implanted implant. The terms “lateral” and “right” or “left” accordingly denote in each case areas in the implanted implant that are located laterally to the median sagittal plane.

FIGS. 1-4 show a first embodiment of the spinous process implant. The implant presents upper and lower support means that are designed as a spring leaf 10. The upper spring leaf 10.1 and the lower spring leaf 10.2 here are parts with identical shape that, however, are arranged with mutual twisting by 180° about the longitudinal middle axis of the implant. The spring leaves 10 have the shape of a longitudinally stretched, flat band that is shape elastic, i.e., it maintains its shape, although it is deformable when exposed to a relatively strong elastic resetting force. The spring leaves 10 are manufactured for this purpose from an appropriate biocompatible material, for example, from a metal, particularly a titanium alloy, or from an appropriate plastic, for example PEEK. The spring leaves 10 present, for example, a length of 15-30 mm, preferably 20-25 mm, a width of 4-8 mm, and a thickness of 0.8-1.2 mm.

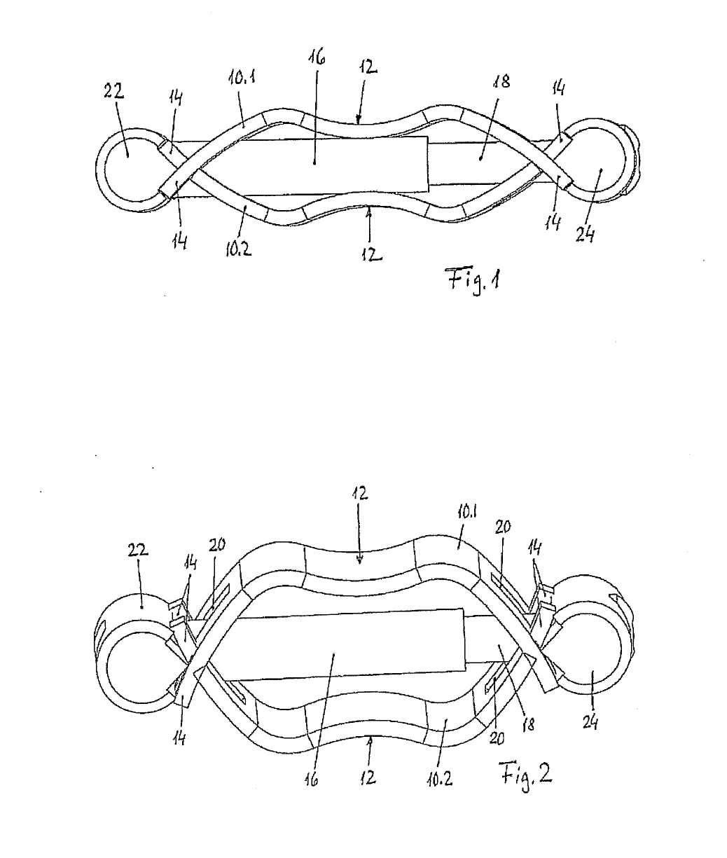

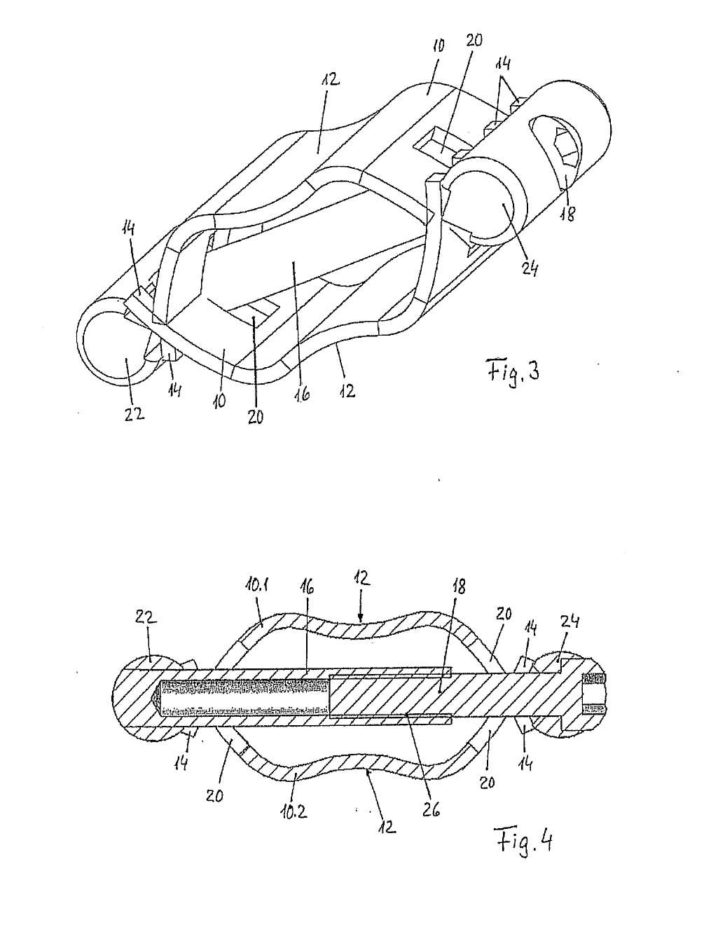

The spring leaves 10 form a convex bulge, upward or downward, in their longitudinal direction, as a flat arc. In the middle area of their longitudinal extent, the spring leaves 10 here present a concave recess with opposite bulge forming a saddle 12. At its two lateral end edges, the spring leaves 10 present notches, so that axially protruding teeth 14 are formed. When the two spring leaves 10.1 and 10.2 are assembled with mutual twisting by 180°, the teeth 14 of the corresponding lateral ends of the two spring leaves 10.1 and 10.2 mutually engage with interdigitation, so that the lateral ends of the two spring leaves 10.1 and 10.2 cross over each other, as can be seen in FIGS. 2 and 3.

Clamping means are inserted between the two spring leaves 10.1 and 10.2, in the middle longitudinal axis of the implant. The clamping means consist of an outer tube 16 and an inner bolt 18 that engages telescopically in the outer tube 16. The outer tube 16 and the inner bolt 18 in each case engage through central axial notches 20 of the lateral end edges of the spring leaves 10. On the mutually facing lateral ends of the outer tube 16 and of the inner bolt 18, an end piece 22 or 24 is arranged in each case. The end pieces 22 and 24 are in the shape of a roller whose axis runs perpendicularly to the axis of the outer tube 16 or of the inner bolt 18. The end pieces 22 and 24 are positioned in each case laterally from outside in the fork of the mutually crossing, interdigitating, lateral ends of the leave springs 10.1 and 10.2.

In an introduction position of the implant, as shown in FIG. 1, the outer tube 16 and the inner bolt 18 are moved apart from each other laterally outward with the end pieces 22 or 24 in the middle axis of the implant. The spring leaves 10.1 and 10.2 are, as a result, stretched flat due to their elastic shape stability, so that they are applied with the mutually facing inner surfaces of the saddle 12 against the clamping means, i.e., particularly against the outer tube 16. The entire implant presents, in this introduction position, a height of only approximately 5-7 mm. In this introduction position, the implant is inserted between the spinous processes of two consecutive vertebrae. For this purpose, with the patient in an appropriate position, a percutaneous incision is made unilaterally and parallel to the middle plane of the patient, and the interspinal space between the spinous processes is made accessible. Using an appropriate introduction instrument, the implant is introduced laterally in the medial direction between the spinous processes, and positioned in the interspinal space, as close as possible to the vertebral arch. The implant is positioned here in such a way that the middle axis, i.e., the axis of the clamping means, namely of the outer tube 16 and of the inner bolt 18, runs substantially perpendicularly to the median sagittal plane. The upper spring leaf 10.1 is directed with its saddle 12 against the spinous process of the upper vertebra, and the lower spring leaf 10.2 with its saddle 12 against the spinous process of the lower vertebra.

Then, the clamping means are actuated, by moving the inner bolt 18 and the outer tube 16 coaxially into each other, and as a result the end pieces 22 and 24 are pulled toward each other in the lateral direction. By means of the end pieces 22 and 24 which are to be moved toward each other, the interdigitating lateral ends of the spring leaves 10.1 and 10.2 are pulled toward each other, so that the spring leaves 10.1 and 10.2 form a convex bulge, upward or downward, and are spread apart in the vertical direction. This spread position is shown in FIG. 2. As a result of the spreading apart of the spring leaves 10.1 and 10.2, the latter are pressed against the corresponding spinous processes, with the result of distracting them in the desired way. Because the spinous processes are located in each case in the saddle 12 of the upper or of the lower spring leaf 10, the implant is protected against shifting in the lateral direction substantially by positive locking. In the median sagittal plane, i.e., in the plane that is perpendicular with respect to the clamping means, the implant is secured by the anatomic concavity of the spinous processes.

The clamping of the clamping means can be achieved in different ways. In an embodiment, as shown in FIG. 4, the inner bolt 18 engages with a thread 26 in an inner thread of the outer tube 16. The design of the threads 26 is self-inhibiting here. The end piece 22 can here be connected firmly to the outer tube 16, while the inner bolt 18 passes through the end piece 24 in a way that allows free rotation. The inner bolt 18 is braced axially with a screw head against the end piece 24, and it can be rotated by means of this screw head from the lateral end side to clamp the clamping means. The self-inhibition of the thread 26 is ensured here by the fact that the implanted implant remains in the clamped and spread position. If surgical removal of the implant becomes necessary, then the inner bolt 18 can be rotated out of the outer tube 16, so that the implant returns to the introduction position. Moreover, the implant can be clamped again in a minimally invasive intervention by means of the screw head, if the separation of the spinous processes has increased, for example, as a result of bone loss.

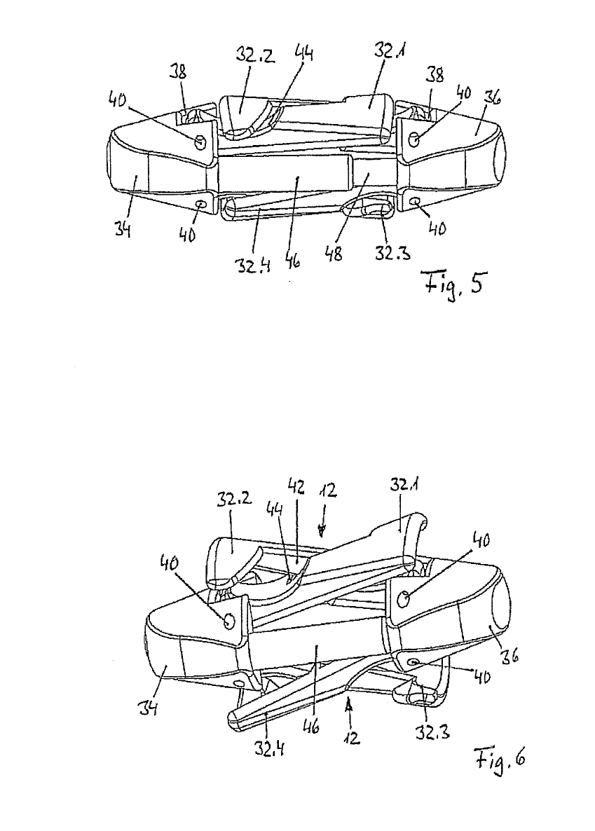

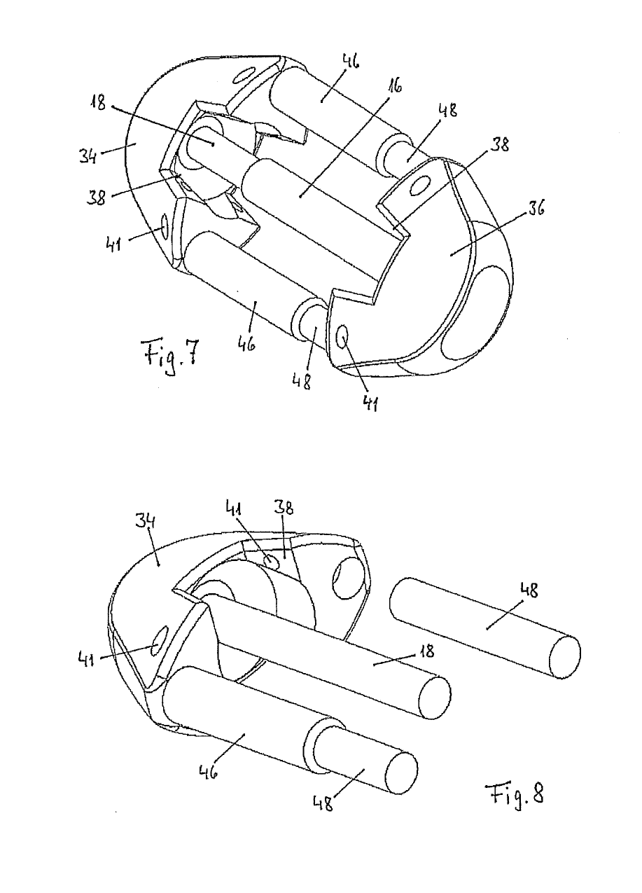

FIGS. 5-9 represent a second embodiment of the spinous process implant.

In this embodiment, the support means are designed as support plates 32. On the upper side, a left support plate 32.1 and a right support plate 32.2 are provided, and, accordingly, on the lower side, a left support plate 32.3 and a right support plate 32.4 are provided. The support plates 32.1, 32.2, 32.3 and 32.4 all have the same shape. The support plates 32 consist of an appropriate biocompatible, dimensionally stable material, for example, metal, particularly a titanium alloy or preferably an appropriate plastic, particularly PEEK. The fact that the shape of the support plates 32.1, 32.2, 32.3 and 32.4 is the same is advantageous with regard to the manufacturing costs.

The support plates 32 present the shape of a plate which is stretched in the longitudinal direction, with a length of approximately 15-30 mm, and a width of approximately 4-8 mm. The thickness of the material is approximately 1-2 mm. The support plates 32 are in each case connected by articulation to lateral end pieces 34 and 36, where the end pieces 34 or 36 can be pulled toward each other in the lateral direction by means of clamping means. The end pieces 34 and 36 here present substantially the same shape of a block.

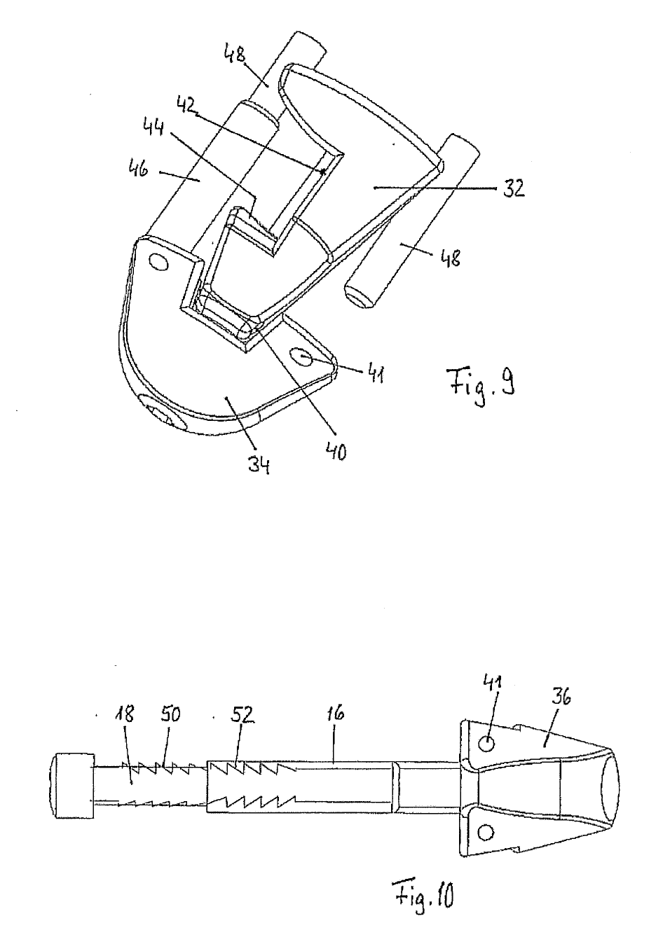

For the swivelable attachment of the support plates 32 on the end pieces 34 or 36, these end pieces 34, 36 present in each case recesses 38, on their mutually facing sides, on the upper side and on the lower side. In each case, an end of a support plate 32 is inserted in these recesses 38, and attached swivelably about a swivel axis 40 which passes through the recess 38, in each case parallel to the upper side or lower side of the implant, and perpendicularly with respect to its middle axis, and which inserted in the bores 41. In this way, the upper left support plate 32.1 is attached swivelably with its left end in the upper recess 38 of the left end piece 34, where the other free end of this support plate 32.1 is directed to the right. Correspondingly, the right upper support plate 32.2 is attached swivelably in an upper recess 38 of the right end piece 36, the left lower support plate 32.3 in a lower recess 38 of the left end piece 34, and the right lower support plate 32.4 in a lower recess 38 of the right end piece 36. The support plates 32 present, at their respective end located in the recess 38, a width that fills the recess 38. The support plates 32 present a greater width at their other free end. In their middle area in the longitudinal direction, the support plates 32 present, in each case on one of their side edges, a lateral cutout 42 which engages up to approximately half of the width of the support plates 32 into the latter. On the end that faces the swivel axis 40, the cutout 42 presents a sliding edge 44 which runs parallel to the swivel axis 40 and thus perpendicularly to the longitudinal extent of the support plate 32. The shape of the support plates 32 can be seen best in FIG. 9. The support plates 32.1 and 32.2 on the upper side, and the support plates 32.3 and 32.4 on the lower side, are in each case mutually twisted by 180° and attached swivelably on the corresponding end pieces 34 or 36. As a result, the left support plate 32.1 and the right support plate 32.2 engage into each other on the upper side, while the left support plate 32.3 and the right support plate 32.4 similarly engage into each other on the lower side, in each case with their cutouts 42. The left support plate 32.1 is positioned with its small middle area in the cutout 42 of the right support plate 32.2, and on the sliding edge 44 of this right support plate 32.2. Conversely, the right support plate 32.2 engages with its middle area in the cutout 42 of the left support plate 32.1, and it is positioned on its sliding edge 44. The lower support plates 32.3 and 32.4 present a corresponding arrangement.

In this second embodiment, the clamping means can also be telescopic clamping means which present an outer tube 16 arranged at one end piece 34, and an inner bolt 18 arranged at the other end piece 36. The clamping means with the outer tube 16 and the inner bolt 18 are arranged in the middle axis of the implant. In the horizontal plane, which is perpendicular to the median sagittal plane, on both sides of the clamping means, guidance means are arranged, which prevent a mutual twisting of the end pieces 34 and 36, and of the support plates 32 attached to the latter, about the middle axis of the clamping means. The guidance means consist, in the embodiment example, in each case of a guidance tube 46 which is arranged on one end piece 34, and a guidance rod 48 which is arranged on the other end piece 36. The guidance rods 48 are guided in each case with axial sliding into the guidance tubes 46. The guidance means formed from the guidance tubes 46 and the guidance rods 48 are parallel to the axis of the clamping means, and arranged at the same separation from the latter.

In the introduction position of the implant, which is shown in FIG. 5, the end pieces 34 and 36 are moved laterally apart from each other. The upper support plates 32.1 and 32.2, which mutually interdigitate via their cutouts 42, and also the lower support plates 32.3 and 32.4 which are positioned with interdigitation above their cutouts 42, lie on each other in this introduction position, so that the entire height of the implant in the vertical direction is small, for example, 5-7 mm. In this introduction position, the implant can be introduced unilaterally into the interspinal space between the spinous processes of two consecutive vertebrae, as described above. The implant is positioned in the interspinal space in such a way that the implant is in the lateral direction symmetric with respect to the median sagittal plane, and the upper support plates 32.1 and 32.2 face the upper spinous process, while the lower support plates 32.3 and 32.4 face the lower spinous process.

Subsequently, the clamping means are actuated, by moving, for example, the inner bolt 18 and the external bolt 16 telescopically toward each other. As a result, the end pieces 34 and 36 are pulled together in the lateral direction. The upper support plate 32.1 and 32.2, and accordingly the lower support plates 32.3 and 32.4, are also moved against each other, as a result. In the process, the upper support plates 32.1 and 32.2 slide on the sliding edge 44 of the other support plates 32.2 and 32.1 in each case, and likewise the lower support plates 32.3 and 32.4 slide against each other. In this sliding motion, the free end of the left support plate 32.1 is swiveled upward by the right end piece 36, while, conversely, the free end of the right support plate 32.2 is swiveled upward by the left end piece 34. The free ends of the lower support plate 32.3 and 32.4 are swiveled downward correspondingly by the respective end pieces 36 or 34. This spread position is shown in FIG. 6. As a result of the upward spreading of the free ends of the support plates 32.1 and 32.2 or 32.3 and 32.4, a central recessed saddle 12 forms between these two ends of the support plates. As the implant is spread apart, the support plates 32 that have been swiveled upward are applied against the corresponding spinous processes, distracting the latter. The spinous processes here lie in each case in the saddle 12 formed between the free ends of the upper or of the lower support plates 32, so that the implant is protected against lateral shifting with positive locking on the spinous processes.

The clamping of the clamping means can also occur in the same way in the second embodiment, as explained in reference to the first embodiment of FIGS. 1-4. Accordingly, the inner bolt 18 can be rotated into the outer tube 16 with a self-inhibiting thread.

An alternative embodiment of the clamping means is shown in FIG. 10. In this embodiment, the inner bolt 18 is designed with latch teeth 50 on its periphery, which engage in the latching receptacles 52 on the inner periphery of the outer tube 16. The latch teeth 50 and the latching receptacles 52 are designed so that, for the purpose of clamping the clamping means, the inner bolt 18 can be pushed into the outer tube 16, while the latched position prevents the inner bolt 18 from being pulled out of the outer tube 16. The tooth partition of the latch teeth 50 and of the latching receptacles 52 here determines the corresponding spread position of the implant. Naturally, this latch design of the clamping means can also be used with the first embodiment of the implant, shown in FIGS. 1-4.

An embodiment of the clamping means in which the latter are latched in successive clamped positions, as shown, for example, in FIG. 10, allows clamping the implant again, for example, in case of bone loss and bone resorption, by means of a resetting force that pushes the inner bolt 18 axially into the outer tube 16, to set the spread position to the next latch position. This resetting force can naturally also be introduced mechanically in a minimally invasive way, as in the case of a resetting by means of a thread. It is also possible to introduce the resetting force by injection, hydraulically or pneumatically or inductively-electrically. An automatic resetting can be achieved by means of an integrated resetting spring force. Such a resetting spring force is applied to the clamping means in the clamping direction, i.e., in the direction in which the support means are spread apart from each other. As long as the support means are applied against the spinous processes, the spinous processes brace the support means against this additional resetting spring force. If the spinous processes yield as a result of bone loss, then the resetting spring force becomes effective, and actuates the clamping means in such a way that the support means are spread apart again, and are applied again against the spinous processes.

The resetting spring force can be achieved, for example, by spring means that are integrated in the clamping means, or also possibly in the guidance means in the embodiment of FIGS. 5-9. Such spring means can consist, for example, of a traction spring which is inserted into the outer tube 16 or the guidance tubes 46, and engages on the inner bolt 18 or the guidance rods 48, pulling the inner bolt 18 into the outer tube 16 or the guidance rods 48 into the guidance tubes 46. Alternatively, pressure springs can be provided, which press the inner bolt 18 into the outer tube 16 or vice versa the outer tube 16 over the inner bolt 18, or the guidance rods 48 and the guidance tubes 46 press into each other in a corresponding way.

Such spring means can consist particularly of a shape memory alloy. Here, the spring means can be kept at a low temperature during the introduction of the implant, at which they are unstressed, and not applied to the clamping means. It is only when the implant has been heated after insertion to the body temperature of the patient that the shape memory spring means go into the state in which they apply the resetting spring force.

Additionally, it should be noted that the surface of the saddle 12, where the upper spring leaf 10.1 is directed with its saddle 12 against the spinous process of the upper vertebra, and the lower spring leaf 10.2 with its saddle 12 against the spinous process of the lower vertebra, presents a surface to the vertebrae which can be adapted to the specific needs of the implant, or be adapted for mass production. The surface can be modified so as to present a coated or cushioned aspect to the biomass to be supported; or, the surface can be manufactured so as to present a non-smooth aspect to the biomass so as to reduce slippage.

Those of skill in the art having studied the current disclosure and appreciated the invention therein will additionally understand that the particular spring forces and spring means elements described herein may be provided by alternative constructions, shapes, and embodiments without departing from the scope and spirit of the present invention. For example the smoothly undulating shape disclosed may be provided by a non-smoothly undulating shape, or one with a series of joined plane portions (not shown) that are effective to result in the purpose achieved by the proposed preferred embodiments. As a result, the present disclosure should be understood to present illustrations for the proposed claims, but the invention is not limited to such illustrations but fully includes the entire scope and spirit of the present invention.

In the claims, means or step-plus-function clauses are intended to cover the structures described or suggested herein as performing the recited function and not only structural equivalents but also equivalent structures. Thus, for example, although a nail, a screw, and a bolt may not be structural equivalents in that a nail relies on friction between a wooden part and a cylindrical surface, a screw's helical surface positively engages the wooden part, and a bolt's head and nut compress opposite sides of a wooden part, in the environment of fastening wooden parts, a nail, a screw, and a bolt may be readily understood by those skilled in the art as equivalent structures.

Having described at least one of the preferred embodiments of the present invention with reference to the accompanying drawings, it is to be understood that the invention is not limited to those precise embodiments, and that various changes, modifications, and adaptations may be effected therein by one skilled in the art without departing from the scope or spirit of the invention as defined in the appended claims.

What is claimed is:

1. A spacer, for use with a spinous process implant, which is positionable with its longitudinal axis substantially perpendicular to the median sagittal plane, between an upper and an adjacent lower spinous process, said spacer comprising:
(a) upper support means for application against said upper spinous process;
(b) lower support means for application against said lower spinous process, and wherein said upper support means and said lower support means can be spread apart between a first introduction setting with a small separation perpendicular to the longitudinal axis, and a second spread position with larger separation perpendicular to the longitudinal axis, in that said upper support means and said lower support means further comprise, in each case:
(i) a recess saddle, by means of which said upper support means and said lower support means are applied in the implanted state against the corresponding spinous process, and, as a result, protected against lateral shifting; and
(ii) clamping means, said clamping means for engaging at the two lateral ends of said upper support means and said lower support means, to spread said upper support means and said lower support means apart, said clamping means located between said upper support means and said lower support means and running in a longitudinal direction.

2. A spacer according to claim 1, wherein the length of said clamping means can be changed telescopically in a longitudinal direction.

3. A spacer according to claim 2, wherein said clamping means further comprises an outer tube and an inner bolt which can be moved coaxially in said outer tube.

4. A spacer according to claim 3, wherein said clamping means are kept in a spread state in locked position by means of a self-inhibiting thread.

5. A spacer according to claim 1, wherein said clamping means further comprise a plurality of end pieces, wherein said plurality of end pieces engage on a lateral end of said upper support means and said lower support means, and are pulled together in a lateral direction to spread said upper support means and said lower support means apart.

6. A spacer according to one of claim 1, wherein said upper support means and said lower support means comprise at least one spring leaf which extends in a longitudinal direction, and which can be spread apart forming a bulge, upward or downward.

7. A spacer according to claim 6, wherein said upper support means and said lower support means further comprise an upper spring leaf and a lower spring leaf, and wherein said upper spring leaf and said lower spring leaf:
(a) have the same design;
(b) are arranged with mutual twisting by 180°;
(c) engage with their lateral end into each other by interdigitation, and
(d) can be spread apart by said clamping means forming a convex bulge, upward or downward.

8. A spacer according to claim 6, wherein said recess saddle is formed by a concavely bulging recess of said at least one spring leaf.

9. A spacer according to claim 1, wherein said upper support means and said lower support means are each formed by a set of one or more support plates that can be swiveled upward.

10. A spacer according to claim 9, wherein said clamping means further comprise a plurality of lateral end pieces, in that said set of one or more support plates is attached swivelably in each case with one end to one of said plurality of end pieces, while the corresponding opposite end can be swiveled upward.

11. A spacer according to claim 10, wherein said set of one or more support plates is shifted on a sliding edge for swiveling upward.

12. A spacer according to claim 11, wherein at least two support plates are provided, wherein a first support plate is attached swivelably to a first end piece, and a second support plate is attached swivelably to a second end piece, and wherein further said first and said second support plates mutually engage by interdigitation with a corresponding lateral cutout, and, in each of said corresponding lateral cutouts, a sliding edge is formed, on which the opposite support plate can be shifted for swiveling upward.

13. A spacer according to claim 12, wherein on each of an upper side and on a lower side of said implant, two support plates are arranged, and said two support plates have the same shape.

14. A spacer according to claim 5, wherein guidance means, for preventing against mutual twisting about the longitudinal direction of said plurality of end pieces, protect said plurality of end pieces during lateral shifting.

15. A spacer according to claim 1, wherein a resetting force in the spreading direction can be applied to said clamping means.

16. A spacer according to claim 15, further comprising spring means for applying a resetting force and wherein said spring means are integrated into said clamping means.

17. A spacer according to claim 16, wherein said spring means are made of a shape memory alloy.

18. The spacer according to claim 14, wherein said guidance means further comprise:
(a) a guidance tube arranged on a corresponding end piece; and
(b) a guidance rod arranged on an opposite end piece, said guidance rod being guidable into a corresponding guidance rod.

19. A method of utilizing a spacer for use with a spinous process implant, which is positionable with its longitudinal axis substantially perpendicular to the median sagittal plane, between an upper and an adjacent lower spinous process, said method further comprising the steps of:
(a) applying upper and lower support means against a corresponding upper and a lower spinous process;
(b) spreading said upper and lower support means apart between a first introduction setting with a small separation perpendicular to the longitudinal axis, and a second spread position with larger separation perpendicular to the longitudinal axis;
(c) utilizing a recess saddle for applying said upper support means and said lower support means, in the implanted state, against said corresponding spinous process; and
(d) engaging, via clamping means the two lateral ends of said upper support means and said lower support means, to spread said upper support means and said lower support means apart, said clamping means located between said upper support means and said lower support means and running in a longitudinal direction.

20. A positionable spacer, for use with a spinous process implant, said spacer comprising:
(a) an upper support means for application against the upper spinous process of a patient;
(b) a lower support means for application against the lower spinous process of a patient, and wherein said upper support means and said lower support means can be spread apart between a first introduction setting with a small separation perpendicular to the longitudinal axis, and a second spread position with larger separation perpendicular to the longitudinal axis, in that said upper support means and said lower support means further comprise, in each case:
(i) a recess saddle, by means of which said upper support means and said lower support means are applied in the implanted state against the corresponding spinous process, and, as a result, protected against lateral shifting; and
(ii) clamping means, said clamping means for engaging at the two lateral ends of said upper support means and said lower support means, to spread said upper support means and said lower support means apart, said clamping means located between said upper support means and said lower support means and running in a longitudinal direction; and
(iii) spring means for applying a resetting force and wherein said spring means are integrated into said clamping means.

21. A positionable spacer, for use with a spinous process implant, said spacer comprising:
(a) Support means for supporting a spinous process, said support means further comprising:
(i) an upper support means for application against the upper spinous process of a patient, and wherein said upper support means further comprises a first set of one or more support plates that can be swiveled upward;
(ii) a lower support means for application against the lower spinous process of a patient, and wherein said lower support means further comprises a second set of one or more support plates that can be swiveled upward; and
wherein said upper support means and said lower support means can be spread apart between a first introduction setting with a small separation perpendicular to the longitudinal axis, and a second spread position with larger separation perpendicular to the longitudinal axis, in that said upper support means and said lower support means further comprise, in each case:
(1) a recess saddle, by means of which said upper support means and said lower support means are applied in the implanted state against the corresponding spinous process, and, as a result, protected against lateral shifting; and
(2) clamping means, said clamping means for engaging at the two lateral ends of said upper support means and said lower support means, to spread said upper support means and said lower support means apart, said clamping means located between said upper support means and said lower support means and running in a longitudinal direction; and
(3) spring means for applying a resetting force and wherein said spring means are integrated into said clamping means.

22. A spacer according to claim 21, wherein said clamping means further comprise a plurality of lateral end pieces, in that said set of one or more support plates is attached swivelably in each case with one end to one of said plurality of end pieces, while the corresponding opposite end can be swiveled upward.

23. A spacer according to claim 21, wherein said set of one or more support plates is shifted on a sliding edge for swiveling upward.

24. A spacer according to claim 22, wherein at least two support plates are provided, wherein a first support plate is attached swivelably to a first end piece, and a second support plate is attached swivelably to a second end piece, and wherein further said first and said second support plates mutually engage by interdigitation with a corresponding lateral cutout, and, in each of said corresponding lateral cutouts, a sliding edge is formed, on which the opposite support plate can be shifted for swiveling upward.

25. A spacer according to claim 21, wherein on each of an upper side and on a lower side of said implant, two support plates are arranged, and said two support plates have the same shape.
